(19) United States
(12) Patent Application Publication
(10) Pub. No.: US 2009/0164014 A1
(43) Pub. Date: Jun. 25, 2009

(54) BIODEGRADABLE OSTOCHONDREAL IMPLANT

(75) Inventors: Elisabeth Liljensten, Kungsbacka (SE); Anders Persoon, Goteborg (SE)

Correspondence Address: YOUNG & THOMPSON, 209 Madison Street, Suite 500, ALEXANDRIA, VA, 22314, US

(73) Assignee: Artimplant AB, Vastra Frolunda (SE)

(21) Appl. No.: 12/090,868

(22) PCT Filed: Oct. 18, 2006
(86) PCT No.: PCT/SE06/01177

§ 371 Date: Aug. 26, 2008

(30) Foreign Application Priority Data
Oct. 21, 2005 (SE) 0502341-1

(51) Int. Cl. A61F 2/28; A61F 2/08

(52) U.S. Cl. 623/16.11; 623/14.12; 623/23.58

(57) ABSTRACT

A biodegradable osteochondral implant comprises a porous top and a porous bottom section separated by a barrier impermeable to agents that have a detrimental effect on the regeneration of cartilage. The implant or its top section is of rotationally symmetric or parallelepipedal form and comprises a resilient polymer material such as polyurethane urea. Also disclosed is a corresponding sheet material from which implants can be excised, processes of manufacture of the implant and the sheet material, and a method for implanting the osteochondral implant in a recess prepared in a load-bearing surface of a joint.

### FIELD OF THE INVENTION

The present invention relates to a biodegradable osteochondral implant, a method for its manufacture and a method for its implantation.

### BACKGROUND OF THE INVENTION

Damaged articular surfaces with or without damage in the underlying bone, such as an articular surface of the knee, can be restored by transfer of an osteochondral plug from a neighbouring region that bears no or little weight (for a review, see: Cartilage Restoration, Part 2. Techniques, Outcomes, and Future Directions. J Winslow Alford and B J Cole, Am J Sports Med 33:443-460 (2005). A problem with this method is the limited availability of suitable autografts and also donor site morbidity. Another method for restoration is autologous chondrocyte implantation. In this method normal hyaline cartilage is harvested by biopsy and expanded in vitro. Cartilage remaining at the damaged area is removed so as to leave healthy surrounding hyaline cartilage to form stable vertical walls around a preferably circular cartilage-free area. A periosteal patch of a size fitting into the cartilage-free area of the defect is removed from a suitable non-weight bearing site of the bone and then sewn onto the cartilage so as to cover the damaged articular surface. The expanded chondrocytes are then implanted into the defect by means of a syringe. Another option is an osteochondral allograft transplantation from a suitable donor.

More recently various matrix tissue scaffolds have been proposed for the substitution of periostal patches, allowing the in-growth of chondrocytes from neighbouring cartilage and also to transfer cultured cells into the defect.

U.S. Pat. No. 5,876,452 (Athanasiou et al.) discloses a cylindrical biodegradable, porous bioerodible implant device of a synthetic material, such as poly(DL-lactide-co-glycolide), consisting of a bone phase that abuts against the underlying bone for anchoring and a cartilage phase that interfaces with the adjacent layer of articular cartilage. For improved in-growth of cartilage cells the cartilage portion of the matrix contains transforming growth factor-β (TGF-β).

U.S. Patent Appln. No. 2005/0043813 (Kusanagi et al.) discloses an acellular matrix implant for implantation into a cartilage lesion comprising a collageneous, gel-gel, polymer of an aromatic organic acid or a thermo-reversible hydrogel fabricated as a sponge or porous honeycomb scaffold. Also disclosed is a biodegradable sealant of a top or bottom cartilage or bone lesion.

U.S. Patent Appln. No. 2003/0229400 (Masuda et al.) discloses a transplantable osteochondral implant comprising cartilage tissue derived from chondrogenic cells cultured in vitro and having a cell associated matrix, the cartilage tissue being attached to a porous biocompatible support scaffold selected from natural bone, demineralised natural bone, collagen, and bone substitute material.

A polyvinyl alcohol-hydrogel implant for replacing worn-out cartilage surfaces is available on the market under the trade name SaluCartilage™ (SaluMedica, Atlanta, Ga.; www.salumedical.com).

In spite of the various devices and methods for restoration of damaged articular surfaces disclosed in the art there is room for substantial improvement.

### OBJECTS OF THE INVENTION

It is an object of the invention to provide a biodegradable osteochondral implant for use in restoring damaged articular surfaces and, if there is need, also subchondral bone, and a method for making the implant.

It is another object of the invention to provide a method for restoring a damaged articular or other bone surface by use of said implant.

Further objects of the invention will become apparent from the following summary of the invention, the description of preferred embodiments illustrated in a drawing, and the appended claims.

### SUMMARY OF THE INVENTION

The present invention is based on the insight that agents emerging from osseous tissue into which an osteochondral implant is to be anchored, in particular blood cells and fibroblasts, and which have a detrimental effect on the regeneration of cartilage should be barred from reaching the area of cartilage regeneration so as not to interfere therewith.

In this application “biodegrable” comprises all kinds of degradation of an implant or a portion thereof in the living body, such as enzymatic, oxidative, and hydrolytic degradation. Furthermore, in this application, “of the same (polymer) material” relates to the chemical nature of the material but not to its form. “Top” and “bottom” faces or sections etc. relate to their disposition in respect of the bottom of a recess prepared by the surgeon in the bone of a joint for implantation.

According to the present invention is disclosed a biodegradable osteochondral implant of preferably cylindrical, conical or other rotationally symmetric form, comprising a porous top and a porous bottom section separated by a barrier, which is impermeable to agents that have a detrimental effect on the regeneration of cartilage, in particular blood and other cells, preferably also impermeable to molecules of 5000 Dalton and even 100,000 Dalton or more. After implantation the barrier is intended to be restored over time by tissue. Other shapes of the implant, such as cubes or parallelepipeds tailored specific requirements are however also within the scope of the invention. The porosity of the top and bottom sections may be the same or different; it is preferred to be at least 50%, more preferred at least 75%, even more preferred at least 85%, most preferred about 90%. While it is preferred for the top and bottom sections to be of the same polymer material their porosity may differ. It is most preferred for the top and bottom sections and the barrier to be of the same polymer material. The top and bottom sections are integral with the barrier and/or adhesively attached to the barrier. The thickness of the barrier is preferably from about 20 μm to about 1 mm, more preferred from 50 μm to 300 μm, most preferred about 100 μm. The barrier should hinder fibroblast and vascular ingrowth as well as haemoglobin from passing from the bottom section hosted in subchondral bone to the top section hosted in cartilage. An upper pore diameter preventing blood to pass through the barrier is 4 μm. Thus a preferred average upper pore diameter is about 2 μm or less.

The porous top section corresponds to the main cartilage layer and is intended to be disposed accordingly when implanted. The height of the top section, that is, the distance between its top and bottom faces or between its top face and the barrier, varies from about 1 to about 6 mm, depending on the site and conditions for implantation. The width of the implant may vary over a wide range such as from 2 mm to 12 mm and more, in particular from about 5 to about 8 mm. The top section, which is intended to be inflated and finally replaced by cartilage over time, may be made more rigid by inclusion of biodegradable stiffening means. It is within the ambit of the invention to provide the top section of the implant with cultured chondrocytes and/or growth stimulating agents such as transforming growth factor-β (TGF-β), platelet-derived growth factor (PDGF), fibroblast growth factor (FGF) and insulin-like growth factor (IGF). It is also within the ambit of the invention to provide the top section with any of serum, hyaluronic acid, hyaluronate, and derivatives of hyaluronic acid.

The porous bottom section corresponds to the subchondral bone and is intended to be disposed accordingly when implanted. The thickness of the bottom section, that is, the distance between its top and bottom faces or between the barrier and its bottom face, varies from about 1 to about 6 mm and more, such as up to 20 mm, depending on the site and conditions for implantation. The top section and/or bottom section may be made more rigid by inclusion of stiffening means such as those described below. Suitable stiffening means for the bottom section are disclosed below. According to a still other preferred aspect of the invention a mineral that is compatible with natural bone, such as calcium phosphate, is made to adhere to and/or is integrated into the bottom section. Over time the bottom section is intended to be replaced by osseous tissue except for biocompatible but not biodegradable stiffening means that it may comprise, which stiffening means will become integrated in the osseous tissue formed.

Additionally the bottom section may be soaked with an aqueous solution comprising a morphogenic protein such as BMP-2 to BMP-7 and BMP-14 and/or a growth factor such as fibroblast growth factor (FGF), platelet derived growth factor (PDGF), epithelial growth factor (EGF), glioma derived growth factor (GDF) and transforming growth factor β (TGF-β), in particular TGF-β1.

It is preferred for the implant of the invention to include a gliding layer corresponding to lamina splendens disposed on the top face of the top section and bonded to it. The gliding layer has a preferred thickness of 0.01-0.1 mm and a porosity inferior to that of the top section. It is preferred for the gliding layer to allow diffusion of articular fluid into the underlying top section. It is also preferred for the gliding layer to allow cartilage cells to be integrated into it. The gliding layer can be provided by, for instance, applying a polymer solution of a concentration of up to 20% by weight on the top face of the top section; preferably the same polymer or the same type of polymer, in particular polyurethane urea, as that of the top section is used for the gliding layer. Over time the gliding layer is intended to be replaced by normal cartilage.

According to a preferred aspect of the invention the top or bottom section, more preferred the top and bottom sections, most preferred the top and bottom sections and the barrier and even optional additional layers are of a polyurethane urea material. It is important to use a polymer material that, while biodegradable, will preserve its physical structure for extended periods of time to provide physical support for in-growing bone and cartilage cells and for expanded chondrocytes with which it may have been seeded, such as for a year and preferably for two years and even three years or more. It is also possible to use in the invention other biocompatible polymer materials that meet these requirements, such as poly(L-lactic acid) and its co-polymers and D-lactic acid and/or glycolic acid (Y S Nam et al., Polymer 20, 1783-1790 (1999); polyglycolide, poly(L-lactic acid), poly(D,L-lactic acid), poly(D,L-lactide-co-glycolide; poly(ε-carprolactone), (DL-lactide-co-caprolactone), poly(glycolide-co-trimethylene carbonate), poly(dioxanone) (S L Ishaug-Riley et al., Biomaterials 20, 2245-2256 (1999); tyrosine-PEG-derived poly(ether carbonate) (C YU et al., Biomaterials 20, 253-264 (1999); poly(ortho esters), copolymers of β-hydroxybutyric acid and hydrovaleric acid, poly(anhydrides), poly(trimethylene carbonate), poly(iminocarbonates) (J Kohn et al., Biomaterials 12, 292-304 (1991); tyrosine-derived polycarbonate (V Tangpasuthadol et al., Biomaterials 21, 2371-2378 (2001); poly(trimethylene carbonate-ε-caprolactone)-block-poly(p-dioxanone) (J-T Hong et al., J Polym Sci: Polym Chem 43(A), 2790-2799 (2005).

The implant of the invention is intended to be anchored in a recess, in particular a bore, extending from a damaged cartilage area of a joint into the bone. According to a preferred aspect of the invention the implant of the invention is dimensioned so as to make the barrier become disposed at the interface of bone and cartilage in a position mounted in the bore. The recess is preferably of a size so as to fully extend into undamaged cartilage disposed laterally of the damaged area. The height of the top section is adapted so as to make its end face facing the joint substantially level with the surface of surrounding cartilage. The resilient nature of the biodegradable implant material allows the implant to be made somewhat larger than the width(s) of the recess in which it is to be anchored; thereby the implant is held in the recess in a slightly compressed state and thus prevented from coming loose.

According to the present invention is also disclosed a method of manufacture of the implant of the invention, comprising: providing first (top) and second (bottom) elements of a biodegradable polymer material, in particular of polyurethane urea, of about parallelepipedal or other suitable form each having at least one flat end face of same form and size; providing a solvent capable of dissolving the polymer; applying an amount of the solvent on at least one of said end faces sufficient to close the pores and make the surface sticky and/or to apply an adhesive polymer solution in a suitable solvent on at least one of the surfaces so as to make them sticky; disposing the at least one sticky end face in an axial circumferentially mirroring position with the other end face; displacing the top and/or bottom elements towards each other until abutment of their end faces; securing them in that relationship for a time sufficient for bonding; removing the solvent, such as in a vacuum chamber or by soaking in an aqueous medium such as water or a mixture of water and alcohol. A preferred solvent for use with polyurethane urea is dimethyl formamide. Also preferred are methyl formamide, methyl acetamide, dimethyl acetamide and dimethyl sulfoxide. The adhesive is one that provides good bonding; it may be of a curing or non-curing type. It is the adhesive, optionally including portions of the surfaces to which it bonds, that provides the barrier of the implant. A preferred method of removal of water soluble solvents like dimethyl formamide and dimethyl sulphoxide by soaking the implant in water. It is also possible to remove solvent from the implant by storing the implant in a vacuum, in particular in a vacuum of less than 10³ pascal.

Another method comprised by the invention is the manufacture of a bonded sheet material from which the implant can be cut out, the method comprising providing first (top) and second (bottom) sheet elements of a biodegradable polymer material of about parallelepipedal or other suitable form each having at least one flat end face of same form and size; providing a solvent, preferably a solvent capable of dissolving the polymer material, optionally containing an adhesive; applying a layer of the solvent on at least one of the end faces; disposing the end face with the adhesive layer in circumferentially aligned mirroring position with the other end face; displacing the top and/or bottom elements towards each other until abutment of their end faces; securing the elements in that position for a time sufficient for the adhesive to adhere to the other face.

The implant of the invention may be made in any suitable form and size. Most preferred are cylindrical implants. Cylindrical implants ending in a cone, a frustrum of a cone or a hemisphere, possibly with rounded bottom face edges, are also preferred. Their diameter is selected so as to make them fit into recesses, in particular bores, in the bone having a diameter of from 5 mm to 20 mm, in particular of from 8 mm to 12 mm. By cutting (milling) rather than drilling the depression into which the implant is to be inserted into the bone, an implant of any desired form with parallel top and bottom faces can be obtained, substantially in form of a parallelepiped. Such an implant is advantageously cut out from a corresponding sheet material comprising top and bottom layers and a barrier in-between them, which sheet material also forms part of the invention. According to the present invention the cutting (milling) of the recess in the bone and of the implant from the bonded sheet material can be controlled by the same or similar software used in dedicated computer-controlled cutting (milling) apparatus.

The implant of the invention comprising or consisting of a top section, a bottom section, and a barrier disposed between them, may additionally comprise stiffening means. Such stiffening elements may be layers, in particular layers disposed parallel with the barrier and preferably adjacent to the barrier. The stiffening means of the bottom section are intended to provide additional stiffness to the implant to locally mimic the mechanical properties of subchondral cortical bone. The stiffening means may comprise a mineral, such as calcium phosphate, calcium carbonate, and bone meal, in particular bone meal from which most or all organic material has been removed of synthetic, semi-synthetic or natural origin and/or a synthetic biodegradable polymer material. A particular preferred form of stiffening element is a textile or non-woven fabric of biodegradable material. To maintain the porosity of a reinforcing layer the use of an adhesive comprising a polymer solution including a porogenic agent, such as one described in SE 518528, is preferred for forming or attaching the layer to other element(s) of the implant. According to another aspect of the invention is also possible to integrate a particulate and/or fibrous stiffening agent in the open porous polymer material of which the implant is made. In a particularly advantageous manner this is done by slightly modifying the procedure for making an open porous material according to the published U.S. Patent Appln. No. 2004/0077739 A1 (Flodin et al.), which is incorporated herein by reference.

According to another aspect of the invention it is however also possible for the stiffening element of the bottom section to be essentially not biodegradable provided that it is biocompatible and can be incorporated in the bone tissue formed from the implant; a suitable biocompatible but not biodegradable material is titanium. Thus it is possible for the implant of the invention to comprise stiffening elements like titanium filaments, nets, rings, etc. Biocompatible metallic stiffening elements can be integrated into the bottom section of the implant. Advantageously they can be provided with an anchoring means, in particular a flexible anchoring means extending from the lateral wall of the bottom element for locking contact with the lateral wall(s) of the recess.

Stiffening means can also be provided in the top section but they must be either biodegradable or removable by the surgeon after having fulfilled their purpose.

According to the invention is also disclosed a method of restoring cartilage in a damaged joint, comprising: providing a recess in the damaged joint of a form so as to dispose the recess circumference in cartilage, providing an implant of preferably substantially parallelepipedal or rotationally symmetric form comprising a top section, a bottom section, and a barrier disposed between the sections, the form of the sections essentially corresponding to the form of the recess; inserting the implant into the recess with the bottom section first, with the proviso that the recess and the implant are dimensioned so as to make the barrier become disposed at the level of the cartilage/bone interface and the free end of the top portion at the level of the cartilage surface facing the joint. The provision of cultured chondrocytes to the top section of the implant is preferred. Also preferred is the provision of an agent selected from serum, hyaluronic acid, hyaluronate, and derivatives of hyaluronic acid to the top section. In another preferred aspect of the invention an apparatus controlled by a computer is used for the excision of the recess for the implant; the data used for providing the recess or sampled when providing the recess are advantageously used for making the implant for this recess from a sheet of bonded biocompatible material with open bores comprising two such layers bonded by a layer of same or similar material that constitutes a barrier to blood cells and chondrocytes. A resiliently compressible implant is preferably anchored in the recess by providing it in a width somewhat larger than the corresponding width of the recess such as, for instance, a width larger by 5% or 10% and even more, so that it is inserted into the recess in a compressed state and held there by its tendency to expand. Instead or in addition, the implant is fixed in adjacent bone and/or cartilage by any of suture, staple, pin, adhesive, such as fibrin glue, hook means, and combinations thereof.

In essentially the same manner as for restoration of cartilage and bone of a load bearing site in a joint the present invention can also be applied to donor site augmentation, that is, to restore cartilage and bone of a non-load bearing site, such as one from which a periostal patch has been removed.

The invention will now be explained in greater detail by reference to a number of preferred embodiments illustrated in a rough drawing.

### SHORT DESCRIPTION OF THE FIGURES

FIG. 1 is a perspective view of a first embodiment of the implant of the invention;

FIG. 2 is a sectional view through a portion of a bone pertaining to a joint comprising a damaged articular surface;

FIG. 3 is the same view as in FIG. 2 after arranging a bore in the bone for insertion of the implant of FIG. 1;

FIG. 4 is the implant of FIG. 1 inserted in the bore, in the same view as in FIG. 3;

FIGS. 5a to 5e illustrate further embodiments of the implant of the invention, in an axial section;

FIGS. 6 and 7 are axial sections of embodiments of the implant of the invention comprising a stiffening element;

FIG. 6a is a top view of the stiffening element of FIG. 6;

FIG. 8 illustrates a preferred method of manufacture of the implant of the invention;

FIG. 9 is another embodiment of the implant of the invention provided with a bottom section stiffening and retention means; in a lateral perspective view;

FIG. 10 is the implant of FIG. 9, in the same view but with the top and bottom portions and the barrier transparent;

FIG. 11 is a further embodiment of the implant of the invention disposed in an applicator prior to insertion into a recess prepared in a bone, in a perspective view;

FIG. 12 is the embodiment of FIG. 11, implanted and in the same view;

FIG. 13 is a portion of a layered stiffened sheet of resilient biodegradable polymer for excision of implants of the invention, in a transverse section.

### EXAMPLE 1

### Implants

The biodegradable cylindrical osteochondral implant 1 of the invention illustrated in FIG. 1 comprises porous top 2 and bottom 3 sections separated by a barrier 4 of same material impermeable to blood and other cells. In this implant the top 2 and bottom sections 3 are of a resilient biocompatible polyurethane urea material (Artelon®, Artimplant AB, Vastra Frölunda, Sweden) having a porosity of about 90%. The barrier 4 is made of the same polymer but lacks the open pores of sections 2 and 3. It extends over an entire axial section of the implant 1. In the Figures the axial dimension (thickness) of the barrier 4 and barriers of other implants of the invention is exaggerated for reasons of visibility. The thickness of the barrier 4 is about 100 μm.

The implant sections 2 and 3 may be provided or soaked with aqueous solutions of agents promoting restoration of the respective tissue. The top section 2 may be soaked with, for instance, with an aqueous solution of hyaluronic acid and/or an agent promoting chondrocyte growth or release and migration from adjacent healthy cartilage, such as platelet-derived growth factor (PDGF), fibroblast growth factor (FGF) and insulin-like growth factor (IGF). The bottom section 3 may be soaked with, for instance, an aqueous solution of one or more of morphogenic protein such as BMP-2 to BMP-7 and BMP-14, fibroblast growth factor (FGF), platelet derived growth factor (PDGF), epithelial growth factor (EGF), glioma derived growth factor (GDF) and transforming growth factor β (TGF-β), in particular TGF-β1.

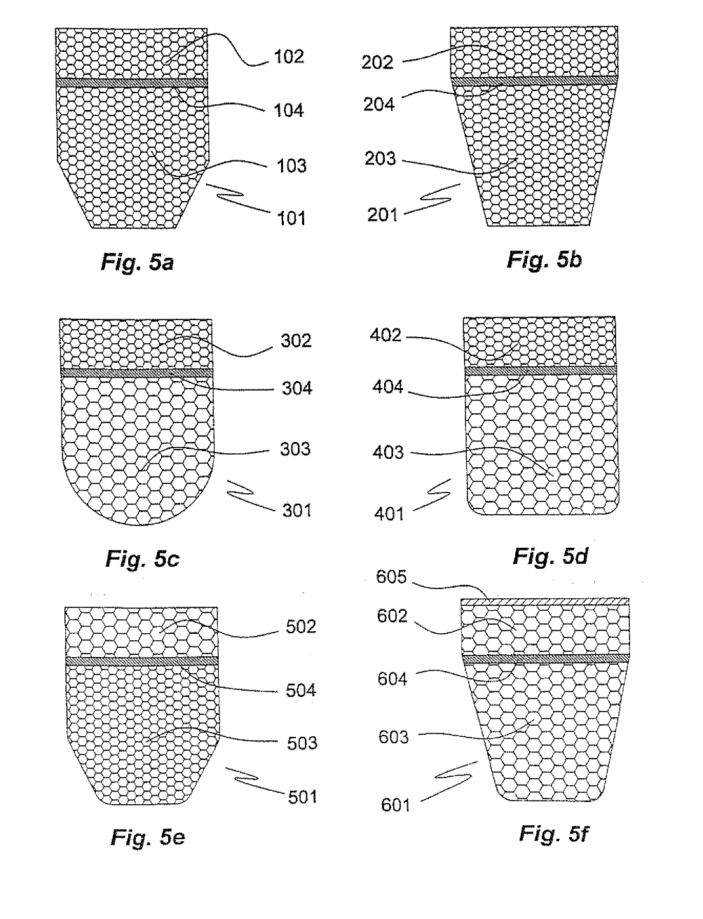

FIGS. 5a-5f represents structural variations 101-601 of the implant of the invention illustrated in FIG. 1. In the implants 101; 201; 601 of FIGS. 5a, 5b, and 5f, the porosity of the top 102; 202; 602 and bottom 103; 203; 603 sections is the same; all of them, including the barriers 104; 204; 604, respectively, are made of the same polymer. In contrast, the top 302; 402; 502 and bottom 303; 403; 503 section pairs of the implants 301-501 of FIGS. 5c-5e differ in their porosity while being made of the same polymer as are the corresponding barriers 204-404. The implants of FIGS. 5a-5f are rotationally symmetric about their long axes of which only the one A-A in FIG. 5a is shown. They are formed to fit into a corresponding bore in the bone of a joint. On its surface facing the joint the top section 602 of the implant of FIG. 5f is provided with a biodegradable protective layer 605 to allow the formation of cartilage in the top section 602 to form undisturbed from movements of the joint, and to provide a temporary joint surface.

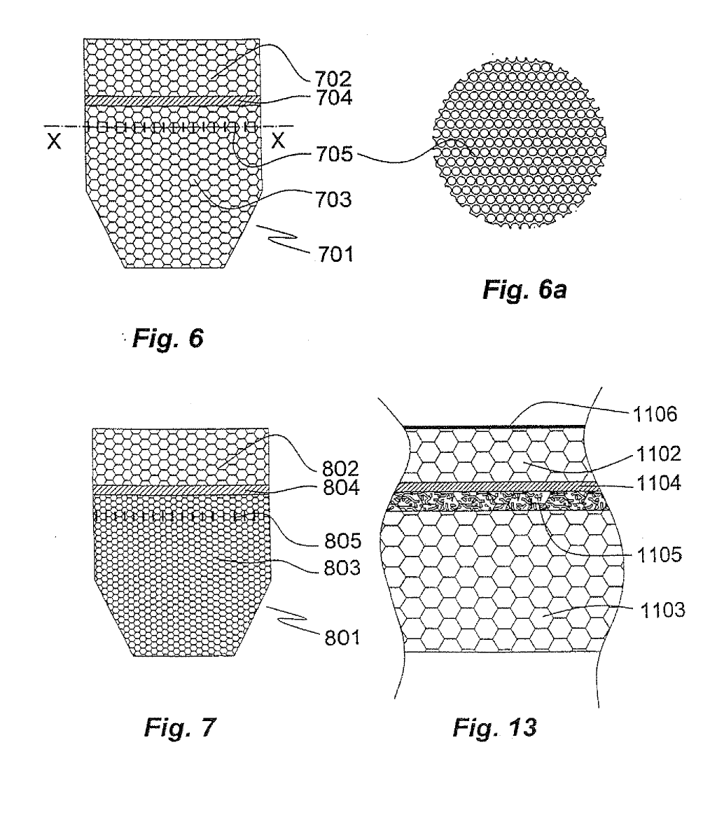

FIGS. 6 and 7 illustrate embodiments 701; 801 of the implant of the invention provided with a stiffening or support element 705; 805 arranged in the bottom section 703, 803 so as to divide the latter into lower and upper subsections. The disposition of the barrier 704; 804 in respect of the bone into which the implants 701 and 801 are intended to be implanted is to make them correspond to the interface between cartilage and subchondral bone. On implantation the stiffening element 705; 805 becomes disposed at about the interface between subchondral bone and skeletal bone but any other disposition in the bottom section is also possible such as, for instance, adjacent to the barrier, in which case the stiffening element has preferably a height comparable to the thickness of the cortical subchondral bone. The stiffening element 705; 805 is of a suitable biodegradable polymer material or a biocompatible but not biodegradable material, preferably the same polymer material as the bottom section, optionally stiffened by inorganic material such as calcium phosphate or carbon fibre. Preferably the stiffening element 705; 805 comprises or consists of a biodegradable textile or non-woven fabric, in particular of the same kind of polymer as the bottom section. It renders physical stability to the bottom section but does not prevent migration of cells etc. between the subsections thereof. The support element 705; 805 of the embodiments in FIGS. 6, 7 is a circular plate (FIG. 6a; section X-X, FIG. 6) disposed perpendicularly in respect of their respective axis of symmetry carrying an array of large (>0.5 mm) through bores separated by thin walls. The top section of embodiments 701 and 801 are designated 702 and 802, respectively. The embodiments of FIGS. 6 and 7 differ in that the porosity of the top and bottom sections is the same (FIG. 6) or different (FIG. 7).

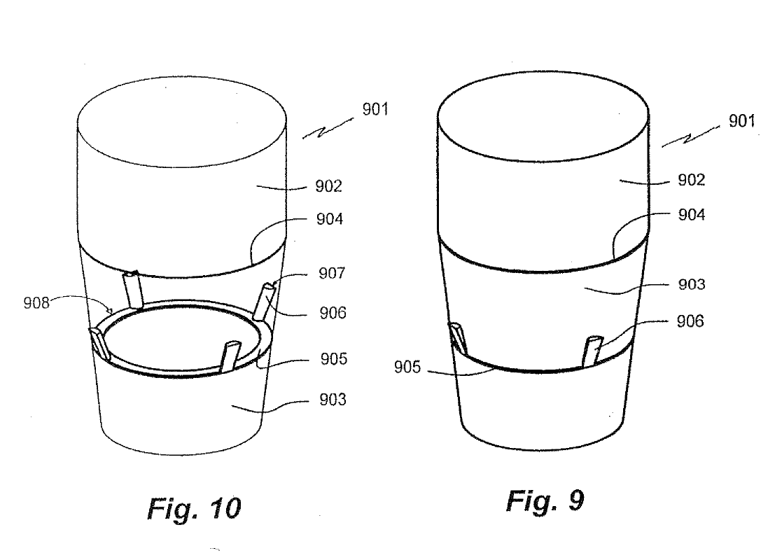

The implant of FIGS. 9 and 10 comprises a cylindrical top section 902 of an open pore polyurethane urea material, a truncated cone bottom section 903 of the same material and a barrier membrane 904 of the same material disposed between the top 902 and bottom 903 sections. An annular stiffening element 905 of titanium disposed in parallel with the barrier membrane 904 is integrated into the bottom section 905 about half-way between the bottom and top faces thereof. The upper and lower outer edges of the stiffening element 905, of which only the upper edge 908 is indicated in FIG. 10, are disposed in the conical surface of the bottom section 903. Four equidistantly spaced tongues 906 integral with the stiffening element 905 extend from its upper face in an upward and outward direction, so as to form an angle with the central axis of the bottom section that is somewhat greater than the corresponding angle formed by the conical walls of the bottom section. Each of their free end sections, which thus extend from the conical side wall of the bottom section 903, comprises a top edge 907.

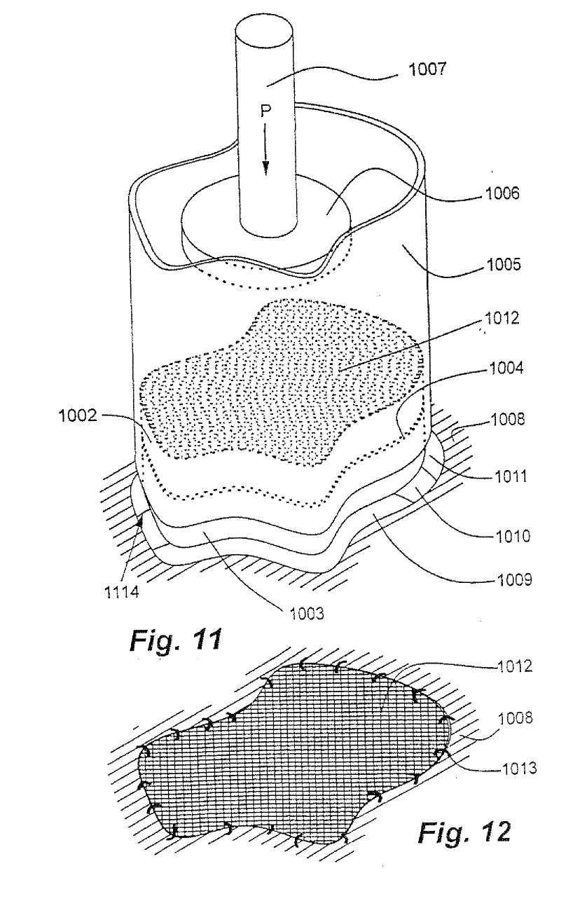

A parallelepipedal but not rotationally symmetric implant 1001 of the invention is shown in FIG. 11. It is made of a viscoelastic polyurethane urea of high porosity (>85%). Its top and bottom sections are designated 1002 and 1003 respectively. The impermeable membrane of same material is designated 1004. The top face 1012 of the implant is provided with a smooth surface.

### EXAMPLE 2

### Method of Implant Manufacture

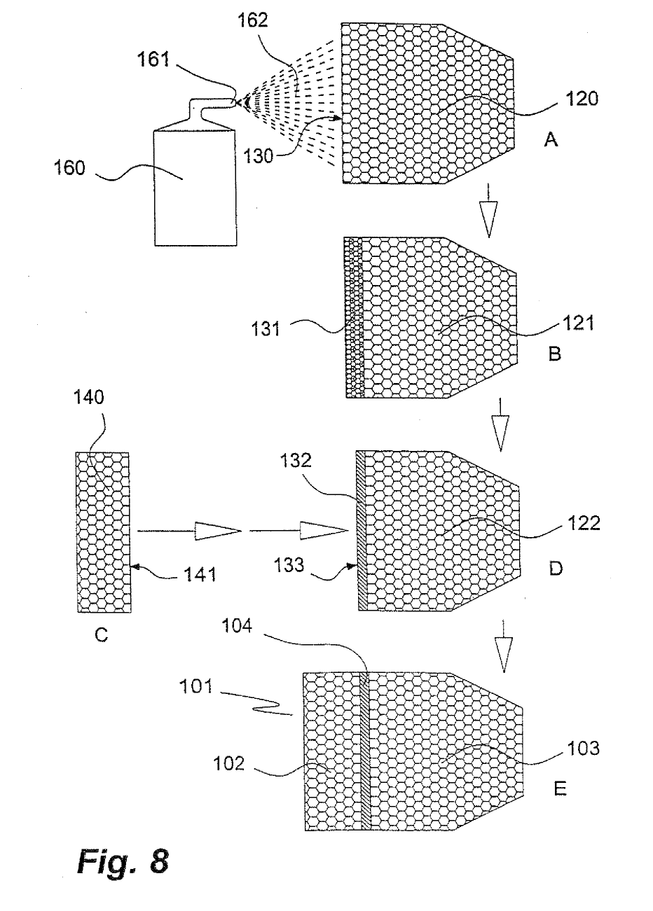

FIG. 8 illustrates a preferred method of manufacture of the implant of FIG. 5a. A bottom section body 120 is cut out from a sheet of highly porous polyurethane urea. In a first step its top face 130 is sprayed with dimethyl formamide (DMF) mist 162 produced by a nozzle 161 of a spray gun 160 (A). Next the DMF on the top face 130 is allowed to penetrate into polyurethane urea matrix under formation of a DMF-containing layer 131 adhering to the remainder 121 of the bottom section 120 (B). In this process the open pores of the layer 131 collapse to form a dense layer 132 shrunken in axial direction adhering to the remainder 122 of the bottom section body (C). A cylindrical top section body 140 cut out from the same material is axially aligned with the remainder 122 of the bottom section body and displace towards the sticky surface 133 of the layer 132 on element 122 (D). On contact of its one circular face 141 with the sticky layer 132 the body 140 becomes adhesively bonded to the layer 132 (E) forming the implant 101 with porous top 102 and bottom 103 sections separated by an impermeable barrier 104 formed from the sticky layer. The barrier 104 lacks open pores and is freed of DMF by water extraction.

In an alternative method of manufacture a bottom section sheet of open porous polyurethane urea is provided. A stiffening fabric sheet of the same material is impregnated with a solution of the same polymer in DMF containing a porogen such as micronized glucose monohydrate. The impregnated stiffening fabric sheet is put on top of the bottom section sheet and a slight pressure is applied to it over its entire surface to evenly attach it to the bottom section sheet. The bonded sheets are then extracted with water to remove porogen and solvent. After drying a thin layer of the same polymer but lacking the porogen is applied on the top face of the stiffening sheet and on the bottom face of a top section sheet cut out from the same material as used for the bottom section sheet. The faces on which polymer solution was applied are put in contact to join the top section sheet and the stiffening fabric sheet bonded to the bottom section sheet to form an implant sheet comprising a barrier impenetrable to cells. The solvent is then extracted from the implant sheet with water and the sheet is dried. The top face of the implant sheet is sprayed with DMF or aqueous DMF mist in a manner similar to that illustrated in FIG. 8 for face 130. This makes the pores on the top face to partly collapse so as to thereby form a smooth gliding layer corresponding to the lamina splendens. The solvent is again extracted with water and the implant sheet dried. A portion of a composite sheet made by this method is shown in FIG. 13. In this figure, 1102 denotes the top section; 1103, the bottom section; 1104, the barrier; 1105, the stiffening layer comprising a mixture of non-woven material of the same kind of polymer as the top and bottom sections 1002, 1003, respectively, and an open pore polymer material of the same kind of polymer as the top and bottom sections; 1106, the gliding layer corresponding to the lamina splendens. From the implant sheet stiffened implants according to the invention can be cut out in any parallelepipedal or rotationally symmetric or other suitable form.

### EXAMPLE 3

### Method of Implantation

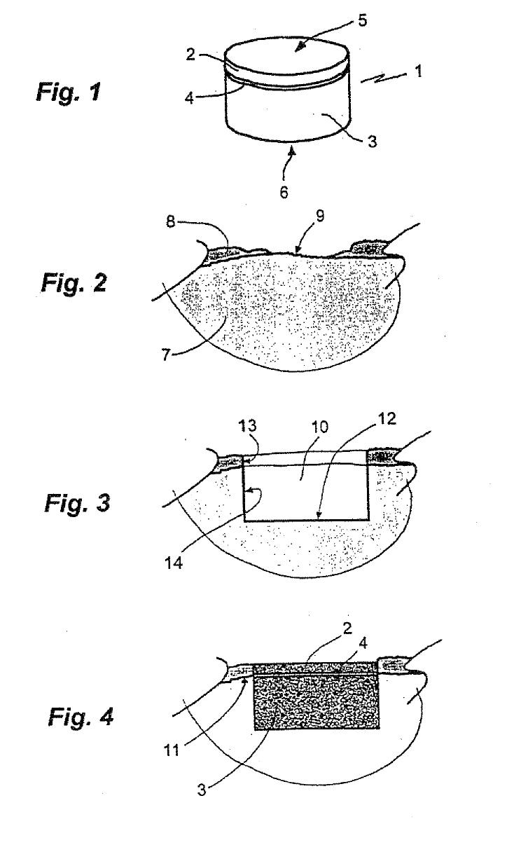

FIG. 2 illustrates a load-bearing portion of a bone 7 pertaining to a joint comprising a damaged articular area 9 substantially free from hyaline cartilage 8. The invention aims at restoring the cartilage in this and similar areas of defective bone surface. For this reason a cylindrical bore 10 is cut into the bone 7 (FIG. 3) covering the entire damaged area 9 and extending into the surrounding cartilage 8 to form a circumferential cartilage wall section 13 extending from the bone in an axial section. The diameter of implant 1 is selected so as to correspond to that of the bore 10. Next the implant 1 is inserted into the bore 10 with free end face 6 of bottom section 3 first until it abuts the bottom 12 of the bore 10. The implant 1 is so dimensioned that, in a mounted state, its barrier 4 becomes disposed at about the level of the cartilage/bone interface 11, while its top face 5 is substantially flush with the surface of the cartilage 8 surrounding the implant 1. The top portion 2 is seeded with autologous condrocytes obtained from a non-load bearing portion of the joint expanded in vitro. The barrier 4 hinders blood cells emerging from blood vessels in the bone 7 from reaching the top section 2 of the implant 1. Thereby conditions for in-growth of the seeded chondrocytes as well as of chondrocytes of the surrounding avascular cartilage 8 into the top section 2 of the implant are substantially improved. Slow biodegradation of the polyurethane urea scaffold of the top section 2 over time allows it to be fully replaced by healthy cartilage so that the damaged joint area is fully restored. Cartilage restoration is paralleled by the ingrowth of osteocytes from the side walls 14 and the bottom 12 of the bore 10 into the polyurethane urea scaffold of the bottom section 3 so as to replace the latter over time by osseous tissue.

Other implants shown in the Figures are designed for implantation into correspondingly adapted bores, which are preferably somewhat more narrow than the respective implant to allow retention thereof in the bore by the resilient nature of the implant material. The implant 901 of FIGS. 9 and 10 is retained by the tongues 906 of the stiffening element 905, which thus also fulfils the role of a retaining element. During insertion of the implant 901 the tongues 906 come into contact with the wall of the bore (not shown) and are deflected inwardly towards the axis of the rotationally symmetric implant. This is possible due to the thin and therefore flexible annulus 905 and the resilient nature of the polymer material in which it is embedded. The sharp edges 907 of the tongues 906 function like hooks and prevent the withdrawal of the implant 901.

Implantation of the implant of FIG. 11 into a recess of corresponding form with a flat bottom 1009 and a side wall perpendicular to the bottom 1009 and comprising a bone section 1010 and a cartilage section 1011 is shown in FIG. 12. Along its entire circumference the recess is disposed in healthy cartilage 1008. The implant is held somewhat compressed (<10%) in a transparent applicator tube 1005 of same cross section. The applicator tube 1005 is of a size allowing it to be inserted into the recess into which the implant 1001 is intended to be inserted. The implant 1001 is expelled from the applicator tube 1005 into the recess by a circular pusher 1006 provided with a shaft 1007 being pushed in the direction P. The applicator tube 1005 is provided with a longitudinal grading for the fine tuning of the expulsion process in which the surgeon may not be able to directly judge the insertion depth of the implant 1001. Upon expulsion into the recess the implant 1001 expands to fill the entire recess until its bottom 1003 and top 1002 sections abut the side wall sections 1010 and 1011, respectively. Thereby the barrier 1004 becomes correctly disposed at the bone/cartilage interface 1014 and the implant 1001 held by the press fit produced by the resilient nature of the implant material in combination with implant widths larger than the corresponding recess widths. It is not necessary that the circumference of the applicator tube 1005 reflects the form of the implant. In most cases a circular applicator tube will work with implants of non-circular form. This is due to the good compressibility of the polyurethane urea material of the invention.

In FIG. 13 the implant 1001 is shown implanted into the recess in which it is additionally anchored by sutures 1013 penetrating its gliding face 1012 and adjacent cartilage 1008.

### EXAMPLE 4

### Integration of a Particulate Calcification-Promoting Agent into an Open Porous Polymer Material for Implants

To a stirred solution of 10 g polyurethane urea prepared according to Example 1a of US 2004/0077739 A1 in DMF was added a mixture of 7.5 g of glucose monohydrate of a mean particle size of about 0.2 mm, 3.0 g of dicalcium hydrogen phosphate dihydrate (mean particle size about 0.1 mm) and 3 g of hydroxyapatite (mean particle size of about 0.05 mm). Stirring was continued for 30 s and the suspension cast on a glass plate to form a thin film, from which the open porous material was obtained in the manner described in Example 1b of US 2004/0077739 A1.

1. Biodegradable osteochondral implant comprising a porous top and a porous bottom section separated by a barrier impermeable to agents that have a detrimental effect on the regeneration of cartilage.

2. The implant of claim 1, wherein the top section or the implant is of parallelepipedal form.

3. The implant of claim 2, of rotationally symmetric form in respect of an axis perpendicular to the barrier.

4. The implant of claim 1, wherein at least one of top section and bottom section comprises a resilient polymer material.

5. The implant of claim 1, wherein the top and bottom sections and the membrane are of the same polymer material.

6. The implant of claim 5, wherein the polymer is polyurethane urea.

7. The implant of claim 4, wherein the polymer comprises open pores providing the top and/or bottom sections with a porosity of 50% or more.

8. The implant of claim 7, wherein the porosity is 75% or more.

9. The implant of claim 7, wherein the porosity is 85% or more.

10. The implant of claim 7, wherein the porosity is about 90%.

11. The implant of claim 1, wherein the barrier is impermeable to liquids.

12. The implant of claim 1, wherein the barrier is impermeable to molecules of 5000 Dalton or more.

13. The implant of claim 1, wherein the barrier is impermeable to molecules of 100,000 Dalton or more.

14. The implant of claim 1, wherein the barrier is impermeable to cells.

15. The implant of claim 1, wherein the top and bottom sections are integral with the barrier and/or adhesively attached to the barrier.

16. The implant of claim 1, wherein the barrier is formed from the top section or the bottom section or both.

17. The implant of claim 1, wherein the thickness of the barrier is from 20 μm to 1 mm.

18. The implant of claim 17, wherein the thickness is from 50 μm to 300 μm.

19. The implant of claim 1, wherein the top section height is from 1 mm to 6 mm.

20. The implant of claim 1, wherein the bottom section height is from 1 mm to 20 mm.

21. The implant of claim 20, wherein the bottom section height is from 1 mm to 6 mm.

22. The implant of claim 1, wherein a mineral compatible with natural bone is made to adhere to and/or is integrated in the bottom section.

23. The implant of claim 1, wherein the bottom sections comprises a retaining element which, while not hindering the insertion of the implant into an implantation recess in a bone, prevents it from being withdrawn.

24. The implant of claim 23, wherein the retaining element is of a biocompatible metal.

25. The implant of claim 23, wherein the retaining element is of a biocompatible polymer that differs from the polymer of the bottom section.

26. The implant of claim 1, comprising a gliding layer disposed on the top face of the top section and bonded to it.

27. The implant of claim 26, wherein the gliding layer has a thickness of 0.01-0.1 mm.

28. The implant of claim 26, wherein the gliding layer has a porosity inferior to that of the top section.

29. The implant of claim 1, cut to standard size and in a sterile package.

30. A method of manufacture of an implant for the reconstitution of cartilage or a layered sheet material for the excision of an implant therefrom, comprising: providing first (top) and second (bottom) elements of a biodegradable polymer material of about parallelepipedal form each having at least one flat end face of same form and size; providing a solvent capable of dissolving the polymer; applying an amount of the solvent on at least one of said end faces sufficient to close the pores and make the surface sticky; disposing the at least one sticky end face in an axial circumferentially mirroring position with the other end face; displacing the top and/or bottom elements towards each other until abutment of their end faces; securing them in that relationship for a time sufficient for bonding; removing the solvent.

31. The method of claim 30, wherein the solvent is removed by soaking the implant in water.

32. The method of claim 30, wherein the polymer material is polyurethane urea.

33. The method of any claim 30, wherein the solvent is selected from methyl formamide, dimethyl formamide, dimethyl sulphoxide, and their mixtures.

34. The method of claim 30, wherein the solvent comprises an adhesive.

35. The method of claim 34, wherein the adhesive comprises the polymer material of the top and/or bottom section.

36. A method of manufacture of an implant for the reconstitution of cartilage, comprising: providing a sheet material according to the method of claim 30; cutting the implant out from the sheet material.

37. A method of restoring cartilage in a damaged joint, comprising: providing a recess in the damaged joint surface of a form so as to dispose the circumference the recess in cartilage, providing an implant of substantially parallelepipedal or rotationally symmetric form comprising a porous top section, a porous bottom section, and a barrier disposed between the sections, the form of the sections essentially corresponding to the form of the recess; inserting the implant into the recess with the bottom section first, with the proviso that the recess and the implant are dimensioned so as to make the barrier become disposed at the level of the cartilage/bone interface and the free end of the top portion at the level of the cartilage surface facing the joint.

38. The method of claim 37, comprising the provision of cultured chondrocytes to the top section of the implant.

39. The method of claim 37, comprising the provision of an agent selected from serum, growth factor, hyaluronic acid, hyaluronate, and derivatives of hyaluronic acid to the top section.

40. The method of claim 37, comprising the use of a drilling or milling apparatus controlled by a computer, wherein data used for providing the recess or sampled when providing the recess is used for making the implant for the recess.

41. The method of claim 37, wherein the bottom section or the top section or both comprise a resiliently compressible matrix, wherein the portion or portions comprising said matrix have a width larger than the corresponding width of the recess, and wherein the implant is inserted into the recess in a laterally compressed state.

42. The method of claim 41, wherein insertion is by means of an applicator tube and a pusher.

43. The method of claim 37, comprising the additional step of anchoring the implant in bone and/or cartilage by one or more of suture, staple, pin, hook means, adhesive.

44. A method of producing a layered stiffened biodegradable sheet material for the excision of an implant therefrom, comprising: providing first and second sheets of a resilient open pore biodegradable polymer material; providing a third sheet of a stiffening biodegradable polymer material in form of a woven or non-woven fabric or a net or a web; providing a first solution of an adhesive polymer in a first solvent in which a porogen is suspended; applying a bonding amount of the first solution on one face of the first sheet and of the third sheet; establishing bonding contact of the faces on which the first solution had been applied to form a first composite sheet; soaking the first composite sheet with an aqueous solvent and drying it; applying a solution of an adhesive polymer in a second solvent on the top face of the fabric or net layer of the first composite sheet and on the bottom face of the second sheet in amounts sufficient to make the open pores coalesce; establishing bonding contact between the faces on which the second polymer solution was applied to form a second composite sheet; soaking the second composite sheet with an aqueous solvent to remove the second solvent to form a layered stiffened biodegradable sheet material for excision of implants; optionally drying the material for excision of implants.

45. The method of claim 44, wherein the biocompatible material is polyurethane urea.

46. The method of claim 44, wherein a barrier impermeable to cells is formed by the coalescence.

47. The method of claim 44, wherein the solvent is selected from methyl formamide, dimethyl formamide, dimethyl sulphoxide, and their mixtures.

48. The method of claim 44, comprising the additional step of spraying the top face of the sheet material for excision of implants with a solvent capable of dissolving the polymer material thereof to form a smooth surface corresponding to the lamina splendens.

49. The method of claim 44, wherein the first and second sheets have an open pore porosity of 50% or more.

50. The method of claim 44, wherein the first sheet has a thickness of from 1 mm to 6 mm, the second sheet has a thickness of from 2 mm to 10 mm.

51. The method of claim 44, wherein the fabric or net layer of the first composite sheet has a thickness of from 0.5 to 3 mm.
